(19) United States
(12) Patent Application Publication
(10) Pub. No.: US 2007/0105880 A1
(43) Pub. Date: May 10, 2007

(54) PROCESS FOR THE PREPARATION OF ALFUZOSIN

(75) Inventors: Nadkarni Sunil Sadanand, Dist Gandhinagar, Gujarat (IN); Ahire Prakash Bhivsan, Dist Gandhinagar, Gujarat (IN); Joshi Uday Chandrakantrao, Dist Gandhinagar, Gujarat (IN)

Correspondence Address: Mr. Dennis A. Bennett;1333 Red River Drive, Post Office Box 38129, St. Louis, MO, 63138, US

(73) Assignee: TORRENT PHARMACEUTICALS LIMITED, Gujarat (IN)

(21) Appl. No.: 11/557,476

(22) Filed: Nov. 7, 2006

(30) Foreign Application Priority Data
Nov. 8, 2005 (IN) 1229/MUM/2004
Oct. 23, 2006 (IN) 1759/MUM/2006

(51) Int. Cl. A61K 31/517; C07D 405/02

(52) U.S. Cl. 514/266.24; 544/284

(57) ABSTRACT

The present invention relates to a simple process for the preparation of alfuzosin, it's bases and its pharmaceutically acceptable salts thereof.

This application claims the benefit of priority of Indian provisional application No. 1229/MUM/2004 filed Nov. 08, 2005, and Indian provisional application No. 1759/MUM/2006, filed Oct. 23, 2006, the disclosures of which are hereby incorporated by reference as if written herein in their entirety.

### FIELD OF THE INVENTION

The present invention relates to a simple process for the preparation of alfuzosin base and its pharmaceutically acceptable salts thereof.

### BACKGROUND OF THE INVENTION

Alfuzosin is chemically known as (R,S)-N-[3-[(4-amino-6,7-dimethoxy-2-quina-zolinyl)methylamino]propyl]tetrahydro-2-furancarboxamide hydrochloride and can be depicted structurally by Formula I. 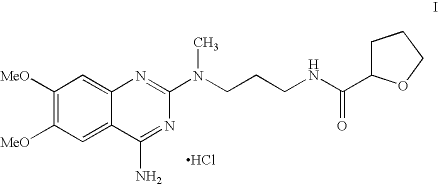
This compound is known to be an antagonist of α₁-adrenergic receptor, and is useful as antihypertensive agent and dysuria curing agent.

Alfuzosin hydrochloride is disclosed in U.S. Pat. No. 4,315,007. The patent also discloses the preparation of alfuzosin hydrochloride reacting 4-amino-2chloro-6,7-dimethoxy quinazoline with 3-methyl amino propionitrile in presence of isoamyl alcohol to obtain N-(4-amino-6,7-dimethoxyquinazol-2yl)-N-methyl-2-cynoethylamine followed by hydrogenation to get N-(4-amino6,7-dimethoxy quinazol-2-yl)-N-methyl propylenediamine. The obtained N-(4-amino6,7-dimethoxy quinazol-2-yl)-N-methyl propylenediamine was treated with activated tetrahydrofuroic acid by adding diamine compound to activated tetrahydrofuroic acid to get residue of alfuzosin base and converting residue of alfuzosin base into hydrochloride salt. The above process is represented by scheme -I.

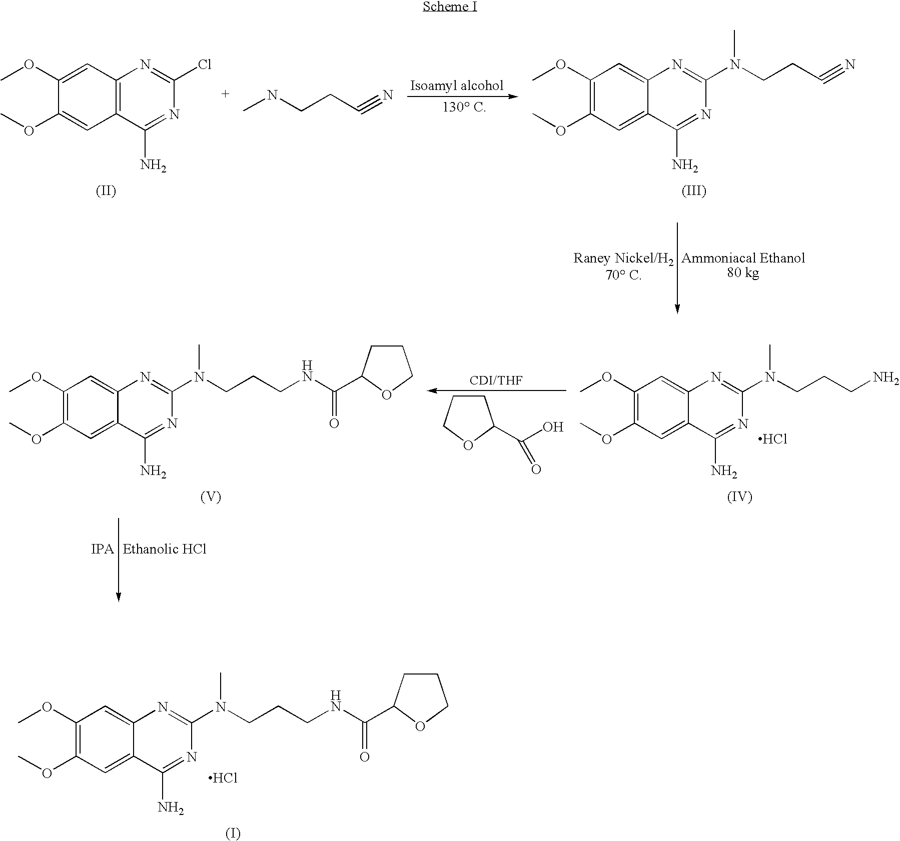

The WO2006/30449 patent application discloses the isolation of albuzosin base and the preparation of alfuzosin hydrochloride by treating N-(4-amino6,7-dimethoxy quinazol-2-yl)-N-methyl propylenediamine with activated tetrahydrofuroic acid by adding activated tetrahydrofuroic acid to diamine compound i.e. N-(4-amino6,7-dimethoxy quinazol-2-yl)-N-methyl propylenediamine, followed by isolating alfuzosin base and converting alfuzosin base into pharmaceutically acceptable salt thereof.

The WO 2006/090268 patent application also discloses the isolation of albuzosin base and the preparation of alfuzosin hydrochloride.

While certain processes of its preparation are known, there is a continuing need for simple and improved processes of preparation of alfuzosin and its salts.

### SUMMARY OF THE INVENTION

In one embodiment, the specification discloses a solid form of Alfuzosin base.

In one embodiment, the specification discloses a process for the preparation of alfuzosin base comprising stirring or dissolving of suspension or crude alfuzosin base in a solvent includes halogenated solvent, aromatic solvent, aliphatic solvent, alcoholic solvent, ester solvent, ketonic solvent, ether solvent; or mixture thereof.

In one embodiment, the specification discloses a process for the preparation of solid of alfuzosin base comprising dissolving alfuzosin base in ketonic solvent, alcoholic solvent or mixture thereof.

In another embodiment, the specification discloses a process for the preparation of alfuzosin base and pharmaceutically acceptable salt thereof comprising reacting 4-amino-2chloro-6,7-dimethoxy quinazoline with 3-methyl amino propionitrile in polar aprotic solvent in presence of base to convert N-(4-amino-6,7-dimethoxyquinazol-2yl)-N-methyl-2-cynoethylamine followed by hydrogenation to obtain diamine compound, followed by treating with activated tetrahydrofuroic acid by adding the diamine compound to activated tetrahydrofuroic acid to obtain a crude alfuzosin base, followed by purification to isolate a alfuzosin base and optionally converting the alfuzosin base into pharmaceutically acceptable salts thereof.

In another embodiment, the specification discloses a process for the preparation of alfuzosin hydrochloride comprising the steps of:
a) reacting 4-amino-2chloro-6,7-dimethoxy quinazoline with 3-methyl amino propionitrile in polar aprotic solvent in presence of base to obtain N-(4-amino-6,7-dimethoxyquinazol-2yl)-N-methyl-2-cynoethylamine;
b) hydrogenating N-(4-amino-6,7-dimethoxyquinazol-2yl)-N-methyl-2-cynoethylamine to obtain dimaine compound;
c) reacting dimaine compound with activated tetrahydrofuroic acid by adding activated tetrahydrofuroic acid to the diamine compound or vice versa without isolating alfuzosin base and converting into alfuzosin hydrochloride.

### DETAILED DESCRIPTION OF THE INVENTION

The term “diamine” refers to N-(4-amino6,7-dimethoxy quinazol-2-yl)-N-methyl propylenediamine.

The term activated tetrahydrofuroic acid refers to tetrahydro-2-furoic acid having its carboxylic acid group in an activated form.

The term “inoculating” has the same meaning as the term “seeding,” and means adding previously obtained solid to facilitate crystallization.

The term “dehydration” means removal of water and/or solvent. Preferably removal of water such that the moisture content is less than 0.8%.

The term “pharmaceutically acceptable salt,” as used herein, represents salts or zwitterionic forms of alfuzosin which are water or oil-soluble or dispersible and therapeutically acceptable as defined herein. The salts can be prepared during the final isolation and purification of the alfuzosin product or separately by reacting alfuzosin in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds of the present invention can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present invention contemplates sodium, potassium, magnesium, and calcium salts of the compounds of the compounds of the present invention and the like.

Basic addition salts can be prepared during the final isolation and purification alfuzosin by reacting a carboxy group with a suitable base such as the hydroxide, carbonate, or bicarbonate of a metal cation or with ammonia or an organic primary, secondary, or tertiary amine. The cations of pharmaceutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

### General Synthetic Methods for Preparing Alfuzosin of the Present Invention

The following schemes can be used to practice the present invention.

The process for the preparation of alfuzosin hydrochloride is depicted in reaction Scheme 2, as below:

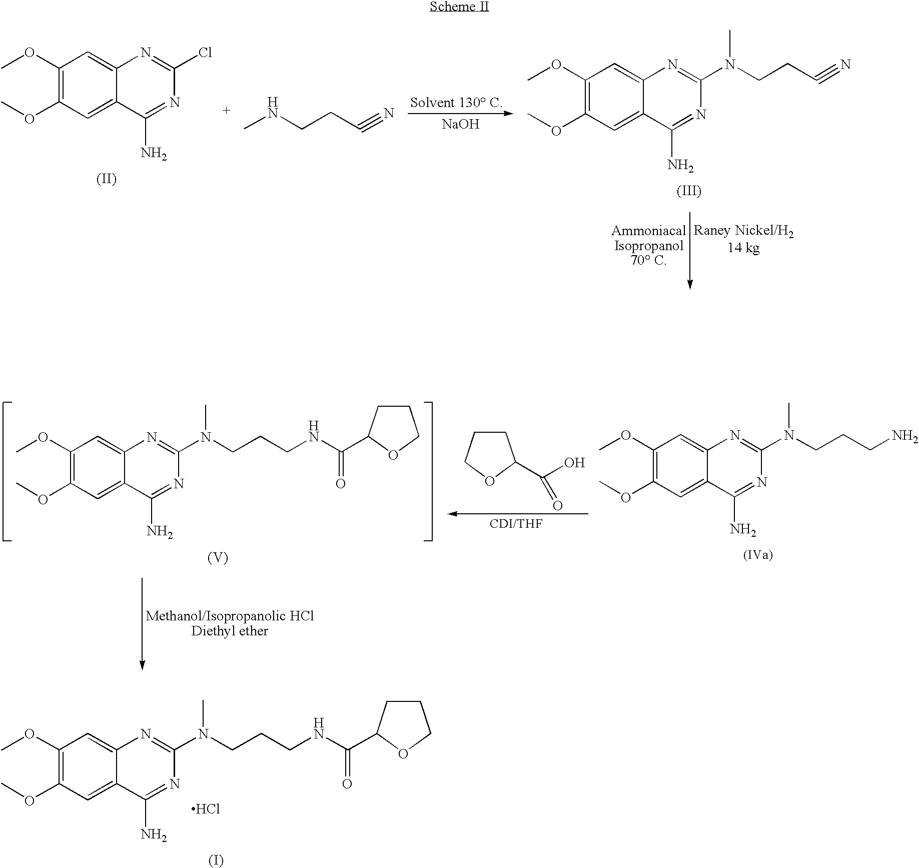

In one embodiment, 4-amino-2chloro-6,7-dimethoxy quinazoline can be reacted with 3-methyl amino propionitrile in polar aprotic solvent in presence of base to obtain N-(4-amino-6,7-dimethoxyquinazol-2yl)-N-methyl-2-cynoethylamine.

The polar aprotic solvent may be selected from toluene, dimethylsulfoxide (DMSO), pyridine, sulfolane, or dichloromethane (DCM) and the like; or mixtures thereof.

The temperature of reaction can range from about 25° C. to reflux temperature of the solvent used.

After cooling the reaction mixture to room temperature, crystalline N-(4-amino-6,7-dimethoxyquinazol-2yl)-N-methyl-2-cynoethylamine compound can be filtered and dried to get crude N-(4-amino-6,7-dimethoxyquinazol-2yl)-N-methyl-2-cynoethylamine compound. After isolation, crude N-(4-amino-6,7-dimethoxyquinazol-2yl)-N-methyl-2-cynoethylamine compound can be purified by treating crude N-(4-amino-6,7-dimethoxyquinazol-2yl)-N-methyl-2-cynoethylamine compound in water followed by addition of base such as potassium or sodium hydroxide, potassium or sodium carbonate, potassium or sodium secondary butoxide, potassium or sodium tertiary butoxide and the like to adjust pH in the range between about 7.25 to 7.50. The addition of water and base can be carried out at room temperature and further cooled to about 9-12° C. or 10° C. and can be dried to get pure N-(4-amino-6,7-dimethoxyquinazol-2yl)-N-methyl-2-cynoethylamine compound.

N-(4-amino-6,7-dimethoxyquinazol-2yl)-N-methyl-2-cynoethylamine prepared according to this embodiment and salts thereof may have a purity greater than 95%,preferably greater than 98% .

N-(4-amino-6,7-dimethoxyquinazol-2yl)-N-methyl-2-cynoethylamine can be further hydrogenated to get diamine compound.

It has been observed that hydrogenation of N-(4-amino-6,7-dimethoxyquinazol-2yl)-N-methyl-2-cynoethylamine can be carried out under a relatively low pressure of about 10-15 Kg within a time period of about 6 hours as compared to prior art process, wherein a pressure of about 80 Kg was employed for about 96 hours to get N-(4-amino6,7-dimethoxy quinazol-2-yl)-N-methyl propylenediamine.

The hydrogenation reaction can be carried out at about 50° C. to 90° C. preferably 65° C. to 75° C. and optionally inoculating with N-(4-amino6,7-dimethoxy quinazol-2-yl)-N-methyl propylenediamine and followed by cooling, filtration and drying to obtain N-(4-amino6,7-dimethoxy quinazol-2-yl)-N-methyl propylenediamine.

The diamine compound can be optionally dehydrated by removal of water from the mixture of diamine compound and solvent. The solvent may be selected from tetrahydrofuran. The diamine compound can be dehydrated under vacuum at 30° C. to 80° C. preferably 40° C.-50° C.

The diamine compound may have moisture of less than about 0.6% preferably less than about 0.3%

The purity of diamine compound i.e. -(4-amino6,7-dimethoxy quinazol-2-yl)-N-methyl propylenediamine may be greater than about 90% preferably about 93%. The yield may be greater than about 75% preferably about 83%.

In one embodiment, the specification discloses a process for the preparation of alfuzosin base and pharmaceutically acceptable salt thereof comprising reacting diamine compound with activated tetrahydrofuroic acid by adding activated tetrahydrofuroic acid to the diamine compound or vice versa with or without isolating a alfuzosin base and converting into pharmaceutically acceptable salt thereof.

It has been surprisingly found that synthesis of alfuzosin base and pharmaceutically acceptable salt thereof can be advantages by reacting diamine compound with activated tetrahydrofuroic acid by adding activated tetrahydrofuroic acid to the diamine compound or vice versa with or without isolating a alfuzosin base and converting into pharmaceutically acceptable salt thereof.

Activated tetrahydrofuroic acid can be prepared by adding tetrahydrofuroic acid and N—N carbonyl di-imidazole or thionyl chloride to tetrahydrofuran under stirring for about 20-40 minutes and cooling the reaction to about −5° C. to −10° C.

In one embodiment, the diamine compound can be added to activated tetrahydrofuroic acid or vice versa to get alfuzosin base. Addition of diamine compound to activated tetrahydrofuroic acid or vice versa can be carried at about −20° C. to 45° C. preferably about 25° C. to 35° C. more preferably about −5° C. to −10° C.

The alfuzosin base may be prepared by adding diamine compound to activated tetrahydrofuroic acid or vice versa in a solvent and treating with an aqueous solution of an inorganic base, preferably sodium hydroxide, the reaction mass obtained is a two-phase system containing an aqueous phase and an organic phase. The organic phase includes solvent and alfuzosin base. The organic phase may be separated and the solvent can be removed partially or completely to provide evaporation residue or solution of alfuzosin freebase in a solvent. Alternatively, the solvent can be added to the evaporation residue to form a solution containing alfuzosin base. The alfuzosin base thus obtained can be converted into salts by addition of acid. The alfuzosin base can also be prepared by adding diamine compound to activated tetrahydrofuroic acid in a solvent and treating with an aqueous solution of an inorganic base, preferably sodium hydroxide; the reaction mass obtained is a two-phase system containing an aqueous phase and an organic phase. The organic phase includes solvent and alfuzosin base. The organic phase may be separated and the solvent removed partially or completely to provide evaporation residue or solution of alfuzosin freebase in a solvent, followed by purification of crude alfuzosin base to isolate pure alfuzosin base.

Alternatively solvent can be added to the evaporation residue to form a solution containing alfuzosin base, the solution optionally seeded with alfuzosin base, followed by purification of crude alfuzosin base and isolating the pure alfuzosin base.

The solvent may be selected from halogenated solvent, aromatic solvent, aliphatic solvent, alcoholic solvent, ester solvent, ketonic solvent, ether solvent; or mixture thereof. The halogenated solvent may be selected from dichloromethane and chloroform; or mixtures thereof. The ether solvent may be diethyl ether, tetrahydrofuran or mixture thereof. The alcoholic solvent may be methanol, ethanol, propanol, isopropanol, butanol, tertiary butyl alcohol or mixture thereof. The ester solvent may be methyl acetate, ethyl acetate, n-propyl acetate, isopropyl acetate, n-butyl acetate; or mixture thereof. The aromatic solvent may be toluene, xylene; or mixture thereof. The ketonic solvent may be acetone, methylethyl ketone, metylsiobutylketone, methylisopropylketone, and methylterbutylketone; or mixture thereof.

The solvent can be removed to obtain crude alfuzosin base by using any methods of drying including distillation with or without vacuum, spray drying, rotational evaporation (such as using a Buchi Rotavapor), agitated thin film drying-vertical (ATFD-V), spin-flash drying, fluid-bed drying, filtration, filtration under vacuum, decantation and centrifugation.

The removal of solvent to prepare alfuzosin base may be carried at temperature depending on solvent used. The temperature can be range from about 20° C. to 130° C. preferably 20° C. to 70° C. more preferably 50° C. to 65° C.

The crude alfuzosin base, as evaporation residue or solution containing alfuzosin base, can be further purified by adding the crude base in a solvent at a temperature from about 20° C. to 150° C. or at the reflux temperature of the solvent used.

Isolation of the pure alfuzosin base can be carried out by using any techniques such as centrifugation, decantation, gravity filtration, vacuum filtration or filtration.

The alfuzosin base obtained may be dried using any technique for example fluidized bed drying, aerial drying, oven drying, or other techniques known in the art. Drying can be conducted at temperatures with or without an application of vacuum. The drying may be carried out under an inert atmosphere, if desired.

The alfuzosin base when isolated as solid may be obtained in the form of crystalline solid, amorphous material or mixtures thereof. The alfuzosin base may have purity greater than about 94.0% preferably about 98% more preferably about 99.4%.

The pharmaceutically acceptable salt of alfuzosin base can be prepared by treating alfuzosin base with an acid in presence of solvent, optionally adding an antisolvent.

The isolated alfuzosin base can be dissolved in a solvent at the temperature ranging from about 10° C.-90° C. preferably about 20° C.-60° C. or reflux temperature of solvent. The solvent can be alocoholic solvent such as methanol, ethanol, propanol, isopropanol, and the like.

The acid used for the preparation of pharmaceutically acceptable salt of alfuzosin base is hydrochloric acid or hydrogen chloride either as gas or as an aqueous solution or as organic solution such as alocoholic solution, for example isopropanolic, methanolic, ethanolic or propanolic solution. The temperature can be maintained at about 10° C. to 45° C. or about 20-40° C. when acid is added.

The temperature during the salt formation may range from about 0° C. to about 150° C. preferably 50° C. to about 70° C.

Antisolvent can be optionally added to solution of alfuzosin hydrochloride to precipitate alfuzosin hydrochloride. Antisolvent may be ether or ester. Suitable ether solvent may be diethyl ether or isopropyl ether. Suitable ester solvent may be ethyl acetate or dimethyl ester. Antisolvent can be optionally added to solution of alfuzosin hydrochloride at a temperature from about 20° C. to about 60° C. preferably 25° C. to about 35° C.

The solid alfuzosin hydrochloride can be dried using different techniques like fluid bed drying, tray drying and rotatory drying techniques with or without application of vacuum and/or under inert conditions. Drying can be conducted at the temperature from about 20° C. to 110° C. preferably 30° C. to 50° C. under vacuum. The drying can be carried out under an inert atmosphere, if desired.

Alfuzosin hydrochloride can be further purified by dissolving the alfuzosin hydrochloride in a solvent and optionally adding antisolvent to precipitate alfuzosin hydrochloride. Suitable solvent may be selected from alcohol or ester or mixture thereof. The alcoholic solvent may be methanol, ethanol, propanol, isopropanol, butanol and the like or mixture thereof. The ester solvent may be methyl acetate, ethyl acetate, n-propyl acetate, isopropyl acetate, n-butyl acetate, or mixture thereof. The dissolution temperature may range from about 10° C. to 130° C. preferably about 20°C. to 40° C. Alcoholic HCl i.e. isoproponolic HCl can be optionally added to the solution of afuzosin hydrochloride to adjust the pH about 2-4. Antisolvent can optionally be added to the solution of afuzosin hydrochloride to precipitate alfuzosin hydrochloride. The antisolvent may be isopropyl ether. Optionally alfuzosin hydrochloride as seeding material can be added in the solution of afuzosin hydrochloride or after addition of the antisolvent in the solution of afuzosin hydrochloride at the temperature about 20° C. to 50° C. preferably 25° C. to 35° C. The alfuzosin hydrochloride can be isolated by filtration, followed by washing and drying.

The alfuzosin hydrochloride may have purity grater than or equal to about 98% or 99%.

The alfuzosin hydrochloride may have mean particle size of less than 250 μm, preferably 100 μm, more preferably 50 μm.

In another embodiment, the specification discloses a process for the preparation of alfuzosin hydrochloride comprising the steps of:
a) reacting 4-amino-2chloro-6,7-dimethoxy quinazoline with 3-methyl amino propionitrile in polar aprotic solvent in presence of base to obtain N-(4-amino-6,7-dimethoxyquinazol-2yl)-N-methyl-2-cynoethylamine;
b) hydrogenating N-(4-amino-6,7-dimethoxyquinazol-2yl)-N-methyl-2-cynoethylamine to obtain diamine compound;
c) reacting diamine compound with activated tetrahydrofuroic acid by adding activated tetrahydrofuroic acid to the diamine compound or vice versa without isolating alfuzosin base and converting into alfuzosin hydrochloride.

In further another embodiment, the specification discloses a process for preparation of alfuzosin hydrochloride comprising reacting 4-amino-2chloro-6,7-dimethoxy quinazoline with 3-methyl amino propionitrile in polar aprotic solvent in presence of base to convert N-(4-amino-6,7-dimethoxyquinazol-2yl)-N-methyl-2-cynoethylamine followed by hydrogenation to obtain diamine compound, followed by treating with activated tetrahydrofuroic acid by adding the diamine compound to activated tetrahydrofuroic acid to obtain a crude alfuzosin base, followed by purification to isolate a pure alfuzosin base and optionally converting the alfuzosin base into hydrochloride salt.

The process for the preparation of alfuzosin hydrochloride of the present invention is simple, eco-friendly, industrially feasible, and economical.

The following examples illustrate the process of preparation of alfuzosin, intermediate or salts thereof and are not intended to limit the scope of the invention.

### EXAMPLE 1

### Preparation of N-(4-AMINO-6,7-DIMETHOZYQUINAZOL-2-YL)-N-METHYL-2-CYANOETHYLAMINE

To 100 g (0.362 moles) of 4-amino-2-chloro-6,7-dimethoxyquinazoline hydrochloride, was added 34.08 g (0.405 moles) of 3-methylaminopropionitrile and 700 ml of sulfolane, and stirred for 5 hours under reflux temperature 130° C. The reaction mass was cooled to room temperature, filtered the crystallized material and dried under vacuum for an hour. The material was washed with 400 ml of isopropanol in a 3-lit Torson beaker, filtered and vacuum dried for an hour. The material was further dried at 45° C.-50° C. for 6 hours. The crude material was then added to 4120 ml of water, stirred for 30 minutes at room temperature and the pH adjusted between 7.25-7.50. The reaction solution was cooled to 10° C. and stirred for an hour at the same temperature. The crystalline material was filtered, washed twice with 200 ml of water and dried to obtain 95 g of N-(4-amino-6,7-dimethoxyquinazol-2-yl)-N-methyl-2-cyanoethylamine.

HPLC Purity: >98%

### EXAMPLE 2

### Preparation of N₁-(4-AMINO-6,7-DIMETHOZYQUINAZOL-2-YL)-N₁-METHYLPROPYLENEDIAMINE

To 10 g (0.0348 moles) of N-(4-amino-6,7-dimethoxyquinazol-2-yl)-N-methyl-2-cyanoethylamine was added 200 ml ammoniacal isopropanol, 10 g of Raney Nickel in an autoclave. The reaction mixture was hydrogenated under a pressure of 10-15 kg under stirring at 450 rpm and a temperature of 70° C. for 6 hours. The reaction mixture was monitored by HPLC and cooled to room temperature. From the reaction mixture, the catalyst and hyflow were filtered off, and the solvent distilled out from the filtrate. To it, 40 ml of water was added and subsequently seeded with 0.1 g of pure intermediate of N₁-(4-amino-6,7-dimethoxyquinazol-2-yl)-N₁-methylpropylenediamine. The material was stirred for an hour at room temperature, and then cooled and further stirred for an hour at 15° C. The crystals were filtered, washed with water and dried to obtain 8.4 g of N₁-(4-amino-6,7-dimethoxyquinazol-2-yl)-N₁-methylpropylenediamine.

HPLC Purity: >93%

### EXAMPLE 3

### Preparation of (RS)-N-[3-[4-AMINO-6,7-DIMETHOZYQUINAZOLIN-2-YL)(METHYL)AMINO ]PROPYL ]TETRAHYDROFURAN-2-CARBOXAMIDE

To 400 ml of tetrahydrofuran was added 8.73 g (0.075 moles) of tetrahydrofuroic acid and 13.02 g (0.08 moles) of carbonyl diimidazole and the reaction mixture was stirred for half an hour and added 19.5 g (0.067 moles) of N₁-(4-amino-6,7-dimethoxyquinazol-2-yl)-N₁-methylpropylenediamine and monitored by HPLC for completion of the reaction. Tetrahydrofuran was distilled from the reaction mass and added 400 ml of dichloromethane to the residue. The dichloromethane solution was washed twice with 200 ml of 2N NaOH solution and 200 ml of 20% sodium chloride solution. The organic layer was dried over sodium sulfate and distilled out dichloromethane completely to obtain a solid residue. To the solid residue, 60 ml of isopropanol was added and heated to reflux for half an hour with stirring. The solution was cooled and stirred for an hour at 15° C. to obtain a crystalline product. The crystalline product was filtered, washed with 60 ml of chilled (10° C.) isopropanol and dried to obtain 18.5 g of crude (RS)-N-[3-[4-amino-6,7-dimethoxyquinazolin-2-yl)(methyl)amino]propyl]tetrahydrofuran-2-carboxamide. The crude product was added to 93 ml of methanol and 93 ml of ethyl acetate at room temperature (28° C. to 35° C.) and stirred for 30 minute at a temperature of 60° C. to 65° C. to get a clear solution. After confirming clarity of the solution, added charcoal and stirred the reaction mass at 60° C. to 65° C. for 30 minute. Filtered off the charcoal while hot through hyflow bed and washed the bed with 7 ml of hot (55° C. to 60° C.) solvent mixture (1:1 Methanol:Ethyl acetate). The solution was cooled to room temperature, further stirred for a couple of hours and seeded with ˜0.2 g of pure (RS)-N-[3-[4-amino-6,7-dimethoxyquinazolin-2yl)(methyl)amino]propyl]tetrahydrofuran-2-carboxamide. The reaction mass was cooled and stirred at 10° C. for an hour. Filtered the crystals, washed with 7 ml of chilled (0° C. to 5° C.) solvent mixture (1:1 Methanol:Ethyl acetate) and dried to obtain 12 g (61.54% w/w) of (RS)-N-[3-[4-amino-6,7-dimethoxyquinazolin-2-yl)(methyl)amino]propyl]tetrahydrofuran-2-carboxamide.

Purity :>99.5%

### EXAMPLE 4

### Preparation of (RS)-N-[3-[4-AMINO-6,7-DIMETHOXYQUINAZOLIN-2-YL)(METHYL)AMINO)]PROPYL ]TETRAHYROFURAN-2-CARBOXAMIDE HYDROCHLORIDE (I)

To 10 g of (RS)-N-[3-[4-amino-6,7-dimethoxyquinazolin-2-yl)(methyl)amino]propyl]tetrahydrofuran-2-carboxamide was added 40 ml of methanol and 10 ml of isopropanolic HCl till pH 3 to 4 to obtain a clear solution. The solution was filtered through hyflow and added drop wise to 350 ml of diethyl ether and was stirred at room temperature (28° C. to 35°C.) for an hour, cooled to 15° C. and further stirred for an hour at that temperature. The product was filtered under nitrogen atmosphere, dried under vacuum to obtain 8.5 g (85.0% w/w) of (RS)-N-[3-[4-amino-6,7-dimethoxyquinazolin-2-yl)(methyl)amino]propyl]tetrahydrofuran-2-carboxamide hydrochloride and stored in a moisture-free environment.

HPLC Purity: 99.5%

### EXAMPLE 5:

### Determination of Particle Size Using Malvern Mastersizer Standard Bench

Alfuzosin Hydrochloride Sample preparation:

In 50 ml clean and dry beaker, 50 mg of the sample was added to 10 ml of dispersant medium (Liquid paraffin light (LR grade):Toluene (HPLC grade) (90:10) and the solution was sonicated with continuous stirring for 1 minute.

Procedure

The sample unit was filled with about 80 ml of dispersant medium and operated the stirrers at 3000 rpm. The optics were aligned and took background measurement. After measurement the sample preparation was added into sample unit with constant monitoring the obscuration. When the obscuration was between 10% and 30% sample addition was stopped. When the obscuration became stable, the measurements were done twice and average particle size (Histogram) was obtained. D₅₀ of Alfuzosin Hydrochloride was found to be 18.66 μm.

### EXAMPLE 6

### Determination of Surface Area Using Smart Sorb 92/93 Surface Area Analyzers

The empty U shape tube was weighed. Then it was filled with approx 2 g sample (Alfuzosin Hydrochloride) with the help of funnel and the arms of the tube was cleaned by tissue paper straw, again the weight of filled sample tube was taken and it was kept in regeneration chamber at 105° C. for 15 minutes. Silicon stopper was fitted on its arms. The sample was allowed to attain the room temperature and the silicon stopper was removed. The sample tube was fitted to the sample holder. Then the sample was allowed to adsorb Nitrogen from the He, N₂ (70:30) at liquid Nitrogen temperature i.e. −197° C. After completion of the adsorption, sample tube was put in water to get desorption and surface was calculated. The surface area of alfuzosin hydrochloride was found to be 5.13 square meter per gram

### EXAMPLE 7

### (RS)-N-[3-(4-AMINO-6,7-DIMETHOXYQUINAZOLIN-2-YL)METHYLAMINOPROPYL]TETRAHYDROFURAN2CARBOXAMIDE HYDROCHLORIDE

0.1557 Kg (1 mole) of tetrahydrofuroic acid and 0.233 Kg (1 mole) of Carbonyl diimidazole is stirred together in 6.0 liters of tetrahydrofuran to form a reaction mass. This reaction mass is added to 0.35 Kg (1 mole) of the N-(4-amino-6,7-dimethoxyquinazol-2-yl)-N-methylpropylenediamine followed by stirring and later concentrated under vacuum. 7.0 lit. of methylene dichloride is added to the concentrated reaction mass. 1N Sodium hydroxide solution (0.420 kg of NaOH in 10.5 lit. purified water is added and the reaction mass is stirred and allowed to settle. The lower organic layer and upper aqueous layer is separated. The organic layer is washed with purified water and dried with sodium sulphate. The organic layer is filtered and concentrated under vacuum up to 80%. Dry HCl gas is purged into the reaction mass and pH was adjusted to 3 to 4. The reaction mass is filtered though Buchner funnel under vacuum and the residue is suck dried to get (RS)-N-[3-(4-amino-6,7-dimethoxyquinazolin-2-yl)methylaminopropyl]tetrahydrofuran-2-carboxamide hydrochloride.

### EXAMPLE 8

### Preparation of (RS)-N-[3-[4-AMINO-6,7-DIMETHOXYQUINAZONE-2-YL)(METHYL)AMINO]PROPYL]TETRAHYDROFURAN-2-CARBOXAMIDE HYDROCHLORIDE

Part-A: To 250 ml tetrahydrofuran was added 25 g (0.085 moles) of (N-(4-amino-6,7-dimethoxyquinazol-2-yl)-N-methylpropylenediamine) and the reaction mass was distilled till the moisture content was less than 0.3%. The reaction mass was cooled to 0-5° C.

Part-B: To 250 ml tetrahydrofuran was added 17 g (0.146 moles) of tetrahydrofuroic acid and 25 g (0.184 moles) of N—N carbonyl di-imidazole. The reaction mass was cooled to −5 to 10° C. under stirring for 30 minutes.

Part-A was added to Part-B at −5 to −10° C. within 25 to 30 minutes and monitored by HPLC for the completion of reaction. The reaction mass was quenched with mixture of 600 ml of methylene dichloride (MDC) and 250 ml purified water. The aqueous layer was extracted twice with methylene dichloride. The combined organic layer was distilled out completely to obtain solid residue.

To the residue 125 ml of methanol and 125 ml of ethyl acetate were charged and stirred for 30 minutes at 28° C.-35° C. and at 60° C.-65° C. for 1 hour to get a clear solution. Charcoal was added after confirming clarity of solution and the reaction mass was stirred at 60-65° C. for 30 minutes. The charcoal was filtered and hyflowbed was washed with 25 ml of solvent mixture (hot 55° C.-60° C., ethyl acetate-methanol mixture 1:1). Solution was cooled to 25-30° C. and isopropyl alcohol (50 ml) and isopropylonic HCl (40 ml) mixture was added to get the pH 3-4 & stirred at room temp for 30 minutes. 875 ml of diethyl ether was added to reaction mass and stirred at room temp for 30 minutes. The reaction mass was then cooled to 15° C. and further stirred for 1 hour. The product was filtered under nitrogen atmosphere & dried under vacuum. Dried material was purified with mixture of ethyl acetate (125 ml) and methanol (125 ml) and the alfusosin hydrochloride was precipitated in diethyl ether. The obtained alfusosin hydrochloride was then filtered under nitrogen atmosphere & dried under vacuum to obtain 20 g (80% w/w) of (RS)-N-[3-[4-amino-6,7-dimethoxyquinazone-2-yl)(methyl)amino]propyl]tetrahydrofuran-2-carboxamide hydrochloride and stored in a moisture free environment.

### EXAMPLE 9

### Preparation of (RS)-N-[3-[4-AMINO-6,7-DIMETHOXYQUINAZONE-2-YL)(METHYL)AMINO]PROPYL]TETRAHYDROFURAN-2-CARBOXAMIDE HYDROCHLORIDE

Part-A: To 250 ml tetrahydrofuran was added 25 g (0.085 moles) of (N-(4-amino-6,7-dimethoxyquinazol-2-yl) -N-methylpropylenediamine) and the reaction mass was distilled till the moisture content was less than 0.3%. The reaction mass was cooled to 0-5° C.

Part-B: To 250 ml tetrahydrofuran was added 13.6 g (0.117 moles) of tetrahydrofuroic acid and 20 g (0.123 moles) of N—N carbonyl di-imidazole. The reaction mass was then cooled to −5° C. to 10° C. under stirring for 30 minutes.

Part-A was added to Part-B at −5 to −10 C. within 25 to 30 min and monitored by HPLC for the completion of reaction. The reaction mass was quenched with mixture of 600 ml methylenedichloride (MDC) and 250 ml purified water. The aqueous layer was extracted twice with methylenedichloride (MDC). The combined organic layer was distilled out completely to obtain solid residue.

To the residue methylene dichloride 200 ml, 150 ml purified water and 50 ml methanol was charged and the reaction mass was stirred for 30 minutes. To the organic layer 20 g of sodium sulphate was added and moisture content was ensured to be less than 0.5%. Isopropyl alcohol (50 ml) and isopropylonic HCl (50 ml) mixture was added to get the pH 3-4 & stirred at room temp for 30 minutes. The reaction mass was added to 625 ml of diethyl ether and stirred at room temp for 3 hours. The product was filtered under nitrogen atmosphere & dried under vacuum to obtain 25 g (100% w/w) of (RS)-N-[3-[4-amino-6,7-dimethoxyquinazone-2-yl)(methyl) amino]propyl]tetrahydrofuran-2-carboxamide hydrochloride and stored in a moisture free environment.

### EXAMPLE 10

### Preparation of (RS)-N-[3-[4-AMINO-6,7-DIMETHOXYQUINAZONE-2-YL)(METHYL)AMINO]PROPYL]TETRAHYDROFURAN-2-CARBOXAMIDE HYDROCHLORIDE

Part-A: To 250 ml tetrahydrofuran was added 25 g (0.085 moles) of (N-(4-amino-6,7-dimethoxyquinazol-2-yl)-N-methylpropylenediamine) and the reaction mass was distilled till the moisture content was less than 0.3%. The reaction mass was cooled to 0-5° C.

Part-B: To 250 ml tetrahydrofuran was added 13.6 g (0.117 moles) of tetrahydrofuroic acid and 20 g (0.123 moles) of N—N carbonyl di-imidazole. The reaction mass was then cooled to −5 to 10° C. under stirring for 30 minutes.

Part-A was added to Part-B at −5° C. to −10° C. within 25 to 30 min and monitored by HPLC for the completion of reaction. The reaction mass was quenched with mixture of 600 ml of methylene dichloride and 250 ml of purified water. The aqueous layer was extracted twice with methylene dichloride 125 ml. The combined organic layer was distilled out completely to obtain solid residue.

To the residue product 200 ml of methylene dichloride and 125 ml of purified water was charged and stirred the reaction mass for 30 minutes. To the organic layer 20 g sodium sulphate was added and moisture content was ensured to be 0.5%. Isopropyl alcohol (40 ml)+isoproponolic HCl (50 ml) was added to the reaction mass till pH 3-4 is obtained. 125 ml of purified water was charged to reaction mass & aqueous layer was washed with 50 ml of ethyl acetate. 5% of sodium hydroxide solution was added to adjust pH up to 8-9. The reaction mass was extracted with 200 ml of methylene dichloride. 20 g sodium sulphate was added to get the moisture below 0.3%. The organic layer was distilled out completely at 40-45° C. 100 ml of methanol, 40 ml of isopropanolic HCl and 50 ml of isopropyl alcohol was charged at room temperature to get the pH 3-4 & stirred at room temp for 30 min. The reaction mass was added to 625 ml of diethyl ether, stirred at room temp for 30 minutes, cooled to 15° C. and further stirred for 1 hour. The product was filtered under nitrogen atmosphere & dried under vacuum to obtain 10 g (40% w/w) of (RS)-N-[3-[4-amino-6,7-dimethoxyquinazone-2-yl)(methyl)amino]propyl]tetrahydrofuran-2-carboxamide hydrochloride and stored in a moisture free environment. HPLC purity 99.8%

### EXAMPLE 11

### Purification of (RS)-N-[3-[4-AMINO-6,7-DIMETHOXYQUINAZONE-2-YL)(METHYL)AMINO ]PROPYL ]TETRAHYDROFURAN-2-CARBOXAMIDE HYDROCHLORIDE (ALFUZOSIN HYDROCHLORIDE)

0.2 Kg alfuzosin hydrochloride was charged in a clean dry 5 L round bottom flask, 0.8 L methanol was added and the reaction mass was stirred at 25° C.-35° C. 0.380 L isopropanolic HCl was added to the reaction mass at 25° C.-35° C. through addition dropper for 15 minutes to adjust the pH to 3.0-4.0. The reaction mass was stirred and filtered through hyflow on Buckner funnel. The hyflow bed was washed with methanol (0.1 L). The filtrate was collected and kept aside in a closed container. The filtrate was added through addition dropper, to 7.0 L fine filtered diethyl ether, 0.1 g of alfuzosin hydrochloride as seeding material was added to diethyl ether, taken in a clean dry 10 L round bottom flask to facilitate precipitation. The reaction mass was stirred for 1 hr and cooled to 12-15° C. The reaction mass was filtered through a Buckner funnel under nitrogen atmosphere. The wet cake was washed with Diethyl ether (0.6 L) and suck dried; two times. It was weighed and later dried in a vacuum tray drier to obtain pure alfuzosin HCl.

All references, patents or applications, U.S. or foreign, cited in the application are hereby incorporated by reference as if written herein.

From the foregoing description, one skilled in the art can easily ascertain the essential characteristics of this invention, and without departing from the spirit and scope thereof, can make various changes and modifications of the invention to adapt it to various usages and conditions.

We claim:

1. A solid form of Alfuzosin base.

2. Alfuzosin base of claim 1, wherein the purity is at least 95%.

3. Alfuzosin base of claim 2, wherein the purity is at least 99%.

4. A process for the preparation of alfuzosin base comprising stirring or dissolving of suspension or crude alfuzosin base in a solvent.

5. The process of claim 5 wherein the solvent is halogenated solvent, aromatic solvent, aliphatic solvent, alcoholic solvent, ester solvent, ketonic solvent, ether solvent; or mixture thereof.

6. A process for preparation of alfuzosin base or pharmaceutically acceptable salt thereof comprising reacting 4-amino -2chloro-6,7-dimethoxy quinazoline with 3-methyl amino propionitrile in polar aprotic solvent in presence of base to convert N-(4-amino-6,7-dimethoxyquinazol-2yl)-N-methyl-2-cynoethylamine followed by hydrogenation to obtain diamine compound, adding the diamine compound to activated tetrahydrofuroic acid to obtain a crude alfuzosin base, followed by purification to isolate a alfuzosin base and optionally converting the alfuzosin base into pharmaceutically acceptable salts thereof.

7. The process of claim 6, wherein the polar aprotic solvent comprises toluene, dimethylsulfoxide, pyridine, sulfolane, or dichloromethane.

8. The process of claim 6, wherein the base comprises potassium or sodium hydroxide, potassium or sodium carbonate, potassium or sodium secondary butoxide, or potassium or sodium tertiary butoxide.

9. The process of claim 6, wherein hydrogenation is carried out under a low pressure of less than 80 Kg.

10. The process of claim 6, wherein hydrogenation reaction is carried out at 50 to 90° C. or 65 to 75° C.

11. The process of claim 6, wherein hydrogenation further includes seeding with diamine compound.

12. The process of claim 6, wherein the purification is carried out by dissolving the crude alfuzosin base in a solvent selected form halogenated solvent, aromatic solvent, aliphatic solvent, alcoholic solvent, ester solvent, ketonic solvent, ether solvent; or mixture thereof, followed by isolation of alfuzosin base.

13. The process of claim 6, wherein pharmaceutically acceptable salts of alfuzosin base is prepared by reacting the alfuzosin base with an acid in presence of solvent and optionally adding an antisolvent.

14. The process of claim 13, wherein the pharmaceutically acceptable salt of alfuzosin is hydrochloride.

15. The process of claim 13, wherein the acid is hydrochloride acid or hydrogen chloride.

16. The process of claim 13, wherein the solvent is alcohol.

17. The process of claim 13, wherein the antisolvent is ester or ether.

18. A process for the preparation of solid of alfuzosin base comprising dissolving alfuzosin base in ketonic solvent, alcoholic solvent or mixture thereof.

19. The process according to claim 14, wherein the ketonic solvent is methyl isobutylketone.

20. The process according to claim 14, wherein alcoholic solvent is methanol, ethanol.

21. A process for the preparation of alfuzosin hydrochloride comprising the steps of:
a) reacting 4-amino -2chloro-6,7-dimethoxy quinazoline with 3-methyl amino propionitrile in polar aprotic solvent in presence of base to obtain N-(4-amino-6,7-dimethoxyquinazol-2yl)-N-methyl-2-cynoethylamine;
b) hydrogenating N-(4-amino-6,7-dimethoxyquinazol-2yl)-N-methyl-2-cynoethylamine to obtain dimaine compound;
c) reacting dimaine compound with activated tetrahydrofuroic acid by adding activated tetrahydrofuroic acid to the diamine compound or vice versa without isolating alfuzosin base and converting into alfuzosin hydrochloride.

22. The process of claim 21, wherein N-(4-amino6,7-dimethoxy quinazol-2-yl)-N-methyl propylenediamine is dehydrated.

23. The process of claim 21, wherein the polar aprotic solvent comprises toluene, dimethylsulfoxide, pyridine, sulfolane or dichloromethane.

24. The process of claim 21, wherein the base comprises potassium or sodium hydroxide, potassium or sodium carbonate, potassium or sodium secondary butoxide, potassium or sodium tertiary butoxide

25. The process of claim 21, wherein hydrogenation is carried out at pressure of less than 80 Kg.

26. The process of claim 21, wherein hydrogenation reaction is carried out at 50° C. to 90° C. or 65° C. to 75° C.

27. The process of claim 21, wherein hydrogenation further includes seeding with diamine compound i.e. N-(4-amino6,7-dimethoxy quinazol-2-yl)-N-methyl propylenediamine.

28. The process of claim 21, wherein alfuzosin base is provided in a solution of alfuzosin base in a solvent selected form halogenated solvent, aromatic solvent, aliphatic solvent, alcoholic solvent, ester solvent, ketonic solvent, ether solvent; or mixture thereof.

29. The process of claim 21, wherein pharmaceutically acceptable salts of alfuzosin base is prepared by reacting the alfuzosin base with an acid in presence of solvent and optionally adding an antisolvent.

30. The process of claim 29, wherein the pharmaceutically acceptable salt of alfuzosin is hydrochloride salt.

31. The process of claim 29, wherein the acid is hydrochloride acid or hydrogen chloride.

32. The process of claim 29, wherein the solvent is alcohol.

33. The process of claim 29, wherein the antisolvent is ester or ether.

34. Alfuzosin hydrochloride having purity greater than or equal to about 99%.

35. Alfuzosin hydrochloride having mean particle size of less than 100 μm.
